(19) United States
(12) Patent Application Publication
(10) Pub. No.: US 2002/0102642 A1
(43) Pub. Date: Aug. 1, 2002

(54) Monocyte chemotactic protein-5 materials and methods

(75) Inventors: Ronald Godiska, Bothell, WA (US); Patrick W. Gray, Seattle, WA (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(73) Assignee: ICOS Corporation

(21) Appl. No.: 09/872,611

(22) Filed: Jun. 1, 2001

Related U.S. Application Data
(63) Continuation of application No. 08/622,851, filed on Mar. 27, 1996.

(51) Int. Cl. C07K 14/705; C07H 21/04; C12P 21/02; C12N 5/06

(52) U.S. Cl. 435/69.1; 435/320.1; 435/325; 536/23.5; 530/350

(57) ABSTRACT

The present invention provides purified and isolated polynucleotide sequences encoding a novel human macrophage-derived C—C chemokine designated MCP-5. Also provided are purified and isolated chemokine protein, fragments and polypeptide analogs thereof, antibodies thereto, and materials and methods for the recombinant production thereof. These products are useful in therapeutic, diagnostic and medical imaging applications.

The present invention relates generally to chemokines and more particularly to purified and isolated polynucleotides encoding a novel human C—C chemokine designated monocyte chemotactic protein-5 (MCP-5) and analogs thereof, to purified and isolated chemokine polypeptides encoded by the polynucleotides, and to materials and methods for the recombinant production of these polypeptides.

### BACKGROUND OF THE INVENTION

Chemokines, also known as “intercrines” and “SIS cytokines”, comprise a superfamily of small secreted proteins (approximately 70-100 amino acids and 8-10 kiloDaltons in size) which primarily attract and activate leukocytes and thereby aid in the stimulation and regulation of the immune system. The name “chemokine” is derived from the term chemotactic cytokine, and refers to the ability of these proteins to stimulate chemotaxis of leukocytes. Indeed, chemokines may comprise the main attractants for inflammatory cells into pathological tissues. See generally, Baggiolini et al., Advances in Immunology, 55:97-179 (1994). While leukocytes comprise a rich source of chemokines, several chemokines are expressed in a multitude of tissues. Baggiolini et al., supra, Table II. Some chemokines also activate or attract a variety of cell types in addition to leukocytes, such as endothelial cells and fibroblasts.

Previously identified chemokines generally exhibit 20-70% amino acid identity to each other and contain four highly-conserved cysteine residues. Based on the relative position of the first two of these cysteine residues, chemokines have been further classified into two subfamilies. In the “C-X-C” or “α” subfamily, encoded by genes localized to human chromosome 4, the first two cysteines are separated by one amino acid. In the “C—C” or “β” subfamily, encoded by genes on human chromosome 17, the first two cysteines are adjacent. X-ray crystallography and NMR studies of several chemokines have indicated that, in each family, the first and third cysteines form a first disulfide bridge, and the second and fourth cysteines form a second disulfide bridge, strongly influencing the native conformation of the proteins. In humans alone, nearly ten distinct sequences have been described for each chemokine subfamily. Chemokines of both subfamilies have characteristic leader sequences of twenty to twenty-five amino acids.

The C-X-C chemokines, which include IL-8, GROα/β/γ, platelet basic protein, Platelet Factor 4 (PF4), IP-10, and others, share approximately 25% to 60% identity when any two amino acid sequences are compared (except for the GROα/β/γ members, which are 84-88% identical with each other). Most of the subfamily members (excluding IP-10 and Platelet Factor 4) share a common E-L-R tri-peptide motif upstream of the first two cysteine residues. The C-X-C chemokines are generally potent stimulants of neutrophils, causing rapid shape change, chemotaxis, respiratory bursts, and degranulation. These effects are mediated by seven-transmembrane-domain rhodopsin-like G protein-coupled receptors. A receptor specific for IL-8 has been cloned by Holmes et al., Science, 253:1278-83 (1991), while a similar receptor (77% identity) which recognizes IL-8, GRO and NAP2 has been cloned by Murphy and Tiffany, Science, 253:1280-83 (1991). Specific truncation of the N-terminal amino acid sequence of certain C-X-C chemokines, including IL-8, is associated with marked increases in activity.

The C—C chemokines, which include Macrophage Inflammatory Proteins MIP-1α [Nakao et al., Mol. Cell Biol., 10:3646 (1990)] and MIP-1β [Brown et al., J. Immunol., 142:679 (1989)], Monocyte Chemotactic Proteins MCP-1 [Matsushima et al., J. Exp. Med., 169:1485 (1989)], MCP-2 [Van Damme et al., J. Exp. Med., 176:59 (1992) and Chang et al., Int. Immunol., 1:388 (1989)], and MCP-3 [Van Damme et al., supra], RANTES [Schall et al., J. Immunol., 141:1018 (1988)], I-309 [Miller et al., J. Immunol., 143:2907 (1989)], eotaxin [Rothenberg et al., J. Exp. Med., 181:1211-1216 (1995)] and others, share 25% to 70% amino acid identity with each other. This subfamily of chemokines generally activates monocytes, lymphocytes, basophils and eosinophils, but not neutrophils. All of the reported C—C chemokines except eotaxin activate monocytes, causing calcium flux and chemotaxis. More selective effects are seen on lymphocytes, for example, T-lymphocytes, which respond most strongly to RANTES.

Four seven-transmembrane-domain G protein-coupled receptors for C—C chemokines have been cloned to date: a C—C chemokine receptor-1 which recognizes MIP-1α and RANTES [Neote et al., Cell, 72:415-425 (1993)], a receptor for MIP-1α, RANTES and MCP-1 [Power et al., J. Biol. Chem., 270:19495-19500 (1995)], an MCP-1 receptor [Charo et al., Proc. Nat. Acad. Sci., 91:2752-56 (1994)], and an eotaxin receptor [Combadiere et al., J. Biol. Chem. 270:16491-16494 (1995)].

The MCPs are produced by numerous cell types such as fibroblasts, endothelial cells, and mononuclear leukocytes. These cells elaborate chemokines in response to various stimuli such as cytokines, lipopolysaccharide, and infectious agents. The MCPs are thought to be involved in large part in regulating the migration of monocytes to sites of inflammation, where these cells play a role in inflammation, repair and fibrosis. While MCP-1, MCP-2 and MCP-3 share much structural and functional similarity, they also have several distinctive features. MCP-1 production is about ten fold higher than that of MCP-2 and MCP-3 in most cellular systems. [Van Damme et al., J. Immunol., 152:5495-5502 (1994).] The MCP-1 receptor recognizes MCP-1 and MCP-3 but does not bind MCP-2. [Franci et al., J. Immunol., 154:6511-6517 (1995).] The expression patterns of these three MCPs are also distinct.

Both MCP-1 and MCP-3 attract and activate basophils, in addition to recruiting monocytes. MCP-3 has major activities towards eosinophils, while MCP-1 does not activate eosinophils at physiologically relevant concentrations. MCP-2 elicits a weaker migration response in both eosinophils and basophils. [Weber et al., J. Immunol., 154:4166-4172 (1995)]. The isolation of a purported fourth MCP, MCP-4, has been recently described in PCT Publication No. WO 95/31467 dated Nov. 23, 1995.

The role of a number of chemokines, particularly IL-8, has been well documented in various pathological conditions. See generally Baggiolini et al., supra, Table VII. Psoriasis, for example, has been linked to over-production of IL-8, and several studies have observed high levels of IL-8 in the synovial fluid of inflamed joints of patients suffering from rheumatic diseases, osteoarthritis, and gout.

The role of C—C chemokines in pathological conditions has also been documented, albeit less comprehensively than the role of IL-8. For example, the concentration of MCP-1 is higher in the synovial fluid of patients suffering from rheumatoid arthritis than that of patients suffering from other arthritic diseases. The MCP-1 dependent influx of mononuclear phagocytes may be an important event in the development of idiopathic pulmonary fibrosis. The role of C—C chemokines in the recruitment of monocytes into atherosclerotic areas is currently of intense interest, with enhanced MCP-1 expression having been detected in macrophage-rich arterial wall areas but not in normal arterial tissue. MCPs may also be involved in induction of angiogenesis and tumor growth or metastasis. Expression of MCP-1 in malignant cells has been shown to suppress the ability of such cells to form tumors in vivo. (See U.S. Pat. No. 5,179,078, incorporated herein by reference.) Recent evidence also implies that various C—C chemokines may play a role in AIDS treatment. In addition, chemokines may be involved in myelopoiesis. A need therefore exists for the identification and characterization of additional C—C chemokines, particularly MCP chemokines, to further elucidate the role of this important family of molecules in pathological conditions, and to develop improved treatments for such conditions utilizing chemokine-derived products.

Chemokines of the C—C subfamily have been shown to possess utility in medical imaging, e.g., for imaging the site of infection, inflammation, and other sites having C—C chemokine receptor molecules. See, e.g., Kunkel et al., U.S. Pat. No. 5,413,778, incorporated herein by reference. Such methods involve chemical attachment of a labelling agent (e.g., a radioactive isotope) to the C—C chemokine using art recognized techniques (see, e.g., U.S. Pat. Nos. 4,965,392 and 5,037,630, incorporated herein by reference), administration of the labelled chemokine to a subject in a pharmaceutically acceptable carrier, allowing the labelled chemokine to accumulate at a target site, and imaging the labelled chemokine in vivo at the target site. A need in the art exists for additional new C—C chemokines to increase the available arsenal of medical imaging tools.

More generally, due to the importance of chemokines as mediators of chemotaxis and inflammation, a need exists for the identification and isolation of new members of the chemokine family to facilitate modulation of inflammatory and immune responses. For example, substances that promote the immune response may promote the healing of wounds or the speed of recovery from infectious diseases such as pneumonia. Substances that reduce inflammation may be useful for treating pathological conditions mediated by inflammation, such as arthritis, Crohn's disease, and other autoimmune diseases.

Additionally, the established correlation between chemokine expression and inflammatory conditions and disease states provides diagnostic and prognostic indications for the use of chemokines, as well as for antibody substances that are specifically immunoreactive with chemokines; a need exists for the identification and isolation of new chemokines to facilitate such diagnostic and prognostic indications.

For all of the aforementioned reasons, a need exists for recombinant methods of production of newly discovered chemokines, which methods facilitate clinical applications involving the chemokines and/or chemokine inhibitors.

### SUMMARY OF THE INVENTION

The present invention fulfills one or more of the needs outlined above by providing purified and isolated polynucleotides encoding a novel human C—C chemokine designated Monocyte Chemotactic Protein-5 (MCP-5), fragments and analogs thereof, purified and isolated MCP-5 polypeptides, fragments and analogs thereof, materials and methods for the recombinant production of these polypeptides, antibodies to such MCP-5 polypeptides and analogs, and pharmaceutical compositions comprising these polypeptides, fragments, analogs, or antibodies.

The invention specifically provides: purified polynucleotides (i.e., DNA and RNA, both sense and antisense strands) encoding the MCP-5 amino acid sequence of SEQ ID NO: 2, particularly a DNA comprising a nucleotide sequence consisting of the protein-coding portion of the MCP-5 nucleotide sequence of SEQ ID NO: 1; purified polynucleotides encoding amino acids 1 to 75 of SEQ ID NO: 2, particularly a DNA comprising a nucleotide sequence consisting of nucleotides 70 to 297 of SEQ ID NO: 1; and purified polynucleotides encoding a full-length MCP-5 selected from the group consisting of: (a) the DNA of SEQ ID NO: 1; (b) a polynucleotide which hybridizes under stringent conditions to the complementary strand of the DNA of SEQ ID NO: 1 or which would hybridize thereto under stringent conditions but for the degeneracy of the genetic code; and (c) a polynucleotide which encodes the same MCP-5 polypeptide as the DNA of SEQ ID NO: 1. The invention also provides vectors comprising such polynucleotides, particularly expression vectors where DNA encoding MCP-5 is operatively linked to an expression control DNA sequence, host cells stably transformed or transfected with such polynucleotide DNA, and corresponding methods for producing MCP-5 by culturing these host cells and isolating the MCP-5 from the host cells or their nutrient medium. The invention further provides purified MCP-5 polypeptides, particularly a polypeptide comprising the amino acid sequence of SEQ ID NO: 2, or a polypeptide comprising amino acids 1 to 75 of SEQ ID NO: 2. Another aspect of the invention provides antibodies specifically reactive with MCP-5, including monoclonal antibodies and hybridoma cell lines producing such monoclonal antibodies. The invention is described more fully below.

The invention provides purified and isolated polynucleotides (i.e., DNA and RNA, both sense and antisense strands) encoding MCP-5. Preferred DNA sequences of the invention include genomic and cDNA sequences and chemically synthesized DNA sequences.

The nucleotide sequence of a cDNA encoding this MCP-5 chemokine is set forth in SEQ ID NO: 1. A larger cDNA which encodes MCP-5 but which also includes 5′ and 3′ non-coding sequences is set forth in SEQ ID NO: 3. A preferred DNA of the present invention comprises nucleotides 70 to 297 of SEQ ID NO: 1, which comprise the putative coding sequence of the mature, secreted MCP-5 protein without its signal sequence.

The amino acid sequence of chemokine MCP-5 is set forth in SEQ ID NO: 2. Preferred polynucleotides of the present invention include, in addition to those polynucleotides described above, polynucleotides that encode the amino acid sequence set forth in SEQ ID NO: 2, and that differ from the polynucleotides described in the preceding paragraphs only due to the well-known degeneracy of the genetic code.

Similarly, since twenty-three amino acids (positions −23 to −1) of SEQ ID NO: 2 comprise a signal peptide that is cleaved to yield the mature MCP-5 chemokine, preferred polynucleotides include those which encode amino acids 1 to 75 of SEQ ID NO: 2. Thus, a preferred polynucleotide is a purified polynucleotide encoding a polypeptide having an amino acid sequence comprising amino acids 1 to 75 of SEQ ID NO: 2.

Among the uses for the polynucleotides of the present invention is the use as a hybridization probe, to identify and isolate genomic DNA encoding human MCP-5, which gene is likely to have a three exon/two intron structure characteristic of C—C chemokines genes (See Baggiolini et al., supra); to identify and isolate non-human genes encoding proteins homologous to MCP-5; to identify human and non-human chemokines having similarity to MCP-5; and to identify those cells which express MCP-5 and the conditions under which this protein is expressed.

Thus, in another aspect, the invention provides a purified polynucleotide which hybridizes under stringent conditions to the complementary strand of the DNA of SEQ ID NO: 1. Similarly, the invention provides a purified polynucleotide which, but for the redundancy of the genetic code, would hybridize under stringent conditions to the complementary strand of the DNA of SEQ ID NO: 1. Exemplary stringent hybridization conditions are as follows: hybridization at 42° C. in 5X SSC, 20 mM NaPO₄, pH 6.8, 50% formamide; and washing at 42° C. in 0.2X SSC. Those skilled in the art understand that it is desirable to vary these conditions empirically based on the length and the GC nucleotide base content of the sequences to by hybridized, and that formulas for determining such variation exist. [See, e.g., Sambrook et al., Molecular Cloning: a Laboratory Manual. Second Edition, Cold Spring Harbor, New York: Cold Spring Harbor Laboratory (1989).]

In another aspect, the invention includes plasmid and viral DNA vectors incorporating DNAs of the invention, including any of the DNAs described above. Preferred vectors include expression vectors in which the incorporated MCP-5-encoding cDNA is operatively linked to an endogenous or heterologous expression control sequence. Such expression vectors may further include polypeptide-encoding DNA sequences operably linked to the MCP-5-encoding DNA sequences, which vectors may be expressed to yield a fusion protein comprising the MCP-5 polypeptide of interest.

In another aspect, the invention includes a prokaryotic or eukaryotic host cell stably transfected or transformed with a DNA or vector of the present invention. In preferred host cells, the MCP-5 polypeptide encoded by the DNA or vector of the invention is expressed. The DNAs, vectors, and host cells of the present invention are useful, e.g., in methods for the recombinant production of large quantities of MCP-5 polypeptides of the present invention. Such methods are themselves aspects of the invention. For example, the invention includes a method for producing MCP-5 wherein a host cell of the invention is grown in a suitable nutrient medium and MCP-5 protein is isolated from the cell or the medium.

In yet another aspect, the invention includes purified and isolated MCP-5 polypeptides. A preferred peptide is a purified chemokine polypeptide having an amino acid sequence comprising amino acids 1 to 75 of SEQ ID NO: 2. The polypeptides of the present invention may be purified from natural sources, but are preferably produced by recombinant procedures, using the DNAs, vectors, and/or host cells of the present invention, or are chemically synthesized. Purified polypeptides of the invention may be glycosylated or non-glycosylated, water soluble or insoluble, oxidized, reduced, etc., depending on the host cell selected, recombinant production method, isolation method, processing, storage buffer, and the like. Alternatively, MCP-5 polypeptides may be prepared by chemical peptide synthesis using techniques that have been used successfully for the production of other chemokines such as IL-8 [Clark-Lewis et al., J. Biol Chem., 266:23128-34 (1991)] and MCP-1.

The invention also contemplates MCP-5 polypeptide fragments, wherein one or more N-terminal or C-terminal amino acid residues are deleted, and which retain one or more of the biological activities characteristic of the C—C chemokines.

Another aspect of the invention includes MCP-5 polypeptide analogs wherein one or more amino acid residues is added, deleted, or replaced from the MCP-5 of the present invention, and which retain one or more of the biological activities characteristic of the C—C chemokines. Such analogs are useful for, e.g., the medical imaging methods described above or the treatment methods described below. They may be prepared by any recombinant or synthetic methods known in the art, including those described below in Example 6.

A related aspect of the invention includes analogs which lack the biological activities of MCP-5, but which are capable of competitively or non-competitively inhibiting the binding of C—C chemokines with their receptor(s). Such analogs are useful, e.g., in therapeutic compositions or methods for inhibiting the biological activity of endogenous MCP-5 or other C—C chemokines in a host. Such MCP-5 polypeptide analogs are specifically contemplated to modulate the binding characteristics of MCP-5 to chemokine receptors and/or other molecules (e.g., heparin, glycosaminoglycans, erythrocyte chemokine receptors) that are considered to be important in presenting MCP-5 to its receptor.

In related aspects, the invention provides purified and isolated polynucleotides encoding such MCP-5 polypeptide analogs, which polynucleotides are useful for, e.g., recombinantly producing the MCP-5 polypeptide analogs; plasmid and viral vectors incorporating such polynucleotides; and prokaryotic and eukaryotic host cells stably transformed with such DNAs or vectors.

In another aspect, the invention includes antibody substances (e.g., monoclonal and polyclonal antibodies, single chain antibodies, chimeric or humanized antibodies, and the like) which are specifically immunoreactive with MCP-5 polypeptides and polypeptide analogs of the invention. The invention further includes hybridoma cell lines that produce antibody substances of the invention. Such antibodies are useful, for example, for purifying polypeptides of the present invention, for detection or quantitative measurement of MCP-5 in fluid or tissue samples, e.g., using well-known ELISA techniques, and for modulating binding of MCP-5 to its receptor(s). Some chemokine antibodies (e.g., anti-IL-8 antibodies) have been shown to have dramatic anti-inflammatory effects.

Recombinant MCP-5 polypeptides and polypeptide analogs of the invention may be utilized in a like manner to antibodies in binding reactions, to identify cells expressing receptor(s) of MCP-5 and in standard expression cloning techniques to isolate polynucleotides encoding the receptor(s). See, e.g., Example 16 below and the cloning of the IL-8 and MCP-1 receptors in Holmes et al., supra, and Charo et al., supra, respectively. Such MCP-5 polypeptides, MCP-5 polypeptide analogs, and MCP-5 receptor polypeptides are useful for modulation of MCP-5 chemokine activity, and for identification of polypeptide and chemical (e.g., small molecule) MCP-5 agonists and antagonists.

The invention also contemplates pharmaceutical compositions comprising MCP-5 polypeptides, fragments, or analogs thereof for use in methods for enhancing the immune response in a mammal suffering from a wound or an infectious disease. Also contemplated are pharmaceutical compositions comprising MCP-5 polypeptides, fragments, or analogs thereof, or antibodies thereto, for use in methods for reducing inflammation in inflammation-mediated pathological conditions, such as arthritis, Crohn's disease, or other autoimmune diseases. Further contemplated are pharmaceutical compositions for use in reducing atherosclerosis, angiogenesis or tumor growth or metastasis. Such pharmaceutical compositions comprise MCP-5 polypeptide or fragment or analog thereof, or an antibody thereto, with a physiologically acceptable diluent or carrier, and may optionally include other appropriate therapeutic agents, e.g., anti-inflammatory agents. Dosages of the MCP-5 will vary between about 1 μg to 10 mg/kg body weight, depending on the pathological condition to be treated. Such compositions may be administered by a variety of routes depending on the condition to be treated, including via subcutaneous, intramuscular, intravenous, transpulmonary, transdermal, intrathecal, oral, or suppository administration.

The MCP-5 materials and methods described above may be employed in several clinical applications. First, as chemokines attract and activate monocytes and macrophages (Baggiolini et al., supra), MCP-5 expression in a pathogenic inflammatory setting may exacerbate the disease by recruiting additional monocytes and macrophages or other leukocytes to the disease site, by activating the leukocytes that are already there, or by inducing leukocytes to remain at the site. Thus, inhibiting the chemoattractant activity of MCP-5 may be expected to alleviate deleterious inflammatory processes. Significantly, the potential benefits of such an approach have been directly demonstrated in experiments involving IL-8, a C-X-C chemokine that attracts and activates neutrophils. Antibodies directed against IL-8 have a profound ability to inhibit inflammatory disease mediated by neutrophils [Harada et al., J. Leukoc. Biol., 56:559 (1994)]. Inhibition of MCP-5 is expected to have a similar effect in diseases in which monocytes or macrophages are presumed to play a role, e.g., Crohn's disease, rheumatoid arthritis, or atherosclerosis.

Alternatively, augmenting the effect of MCP-5 may have a beneficial role in diseases, as chemokines have also been shown to have a positive effect in wound healing and angiogenesis. Thus, exogenous MCP-5 or MCP-5 agonists may be beneficial in promoting recovery from such diseases.

In addition, the myelosuppressive effect demonstrated for the C—C chemokine MIP-1α (Maze et al., supra) suggests that MCP-5 may have a similar activity. Such activity, provided by MCP-5 or MCP-5 agonists, may yield substantial benefits for patients receiving chemotherapy or radiation therapy, reducing the deleterious effects of the therapy on the patient's myeloid progenitor cells.

MCP-5 or MCP-5 agonists may also prove to be clinically important in the treatment of tumors, as suggested by the ability of the C—C chemokine TCA3 to inhibit tumor formation in mice (see Laning et al., supra). MCP-5 may act directly or indirectly to inhibit tumor formation, e.g., by attracting and activating various non-specific effector cells to the tumor site or by stimulating a specific anti-tumor immunity.

Furthermore, the C—C chemokines RANTES, MIP-1α and MIP-1β have been shown to suppress replication of human immunodeficiency virus HIV-1 [Cocchi et al., Science, 270:1811-1815 (1995)], implicating them as possible therapeutic agents in the prevention or treatment of AIDS. MCP-5's similarity to these chemokines suggests that MCP-5 may also prove to have a role in treating AIDS patients or in preventing onset of the disease.

Additionally, the established correlation between chemokine expression and inflammatory conditions and disease states provides diagnostic and prognostic indications for the use of MCP-5 materials, including antibody substances that are specifically immunoreactive with MCP-5. Such MCP-5 materials are useful in methods for diagnosing and assessing the prognosis of inflammatory conditions and disease states, as well as for medical imaging of areas involved in such conditions and disease states.

Numerous additional aspects and advantages of the invention will become apparent to those skilled in the art upon consideration of the following detailed description of the invention which describes presently preferred embodiments thereof.

### BRIEF DESCRIPTION OF THE DRAWINGS

FIG. 1 is a comparison of the amino acid sequence of human MCP-5 with the amino acid sequences of other, previously characterized human C—C chemokines. A slash “/” marks the site at which putative signal peptides are cleaved. The residues in bold type are conserved within the MCP family. Dashes are inserted to optimize alignment of the sequences.

FIG. 2 shows the effect of MCP-5 and MCP-1 on the monocytic cell line THP-1 in an in vitro chemotaxis assay.

FIGS. 3A and 3B show the effect of MCP-5 and MCP-1 on activation of the monocytic line THP-1. FIGS. 3C and 3D show the effect of these chemokines on activation of a 293 cell line expressing the MCP-1 receptor CCR2-B (Charo et al., supra).

### DETAILED DESCRIPTION

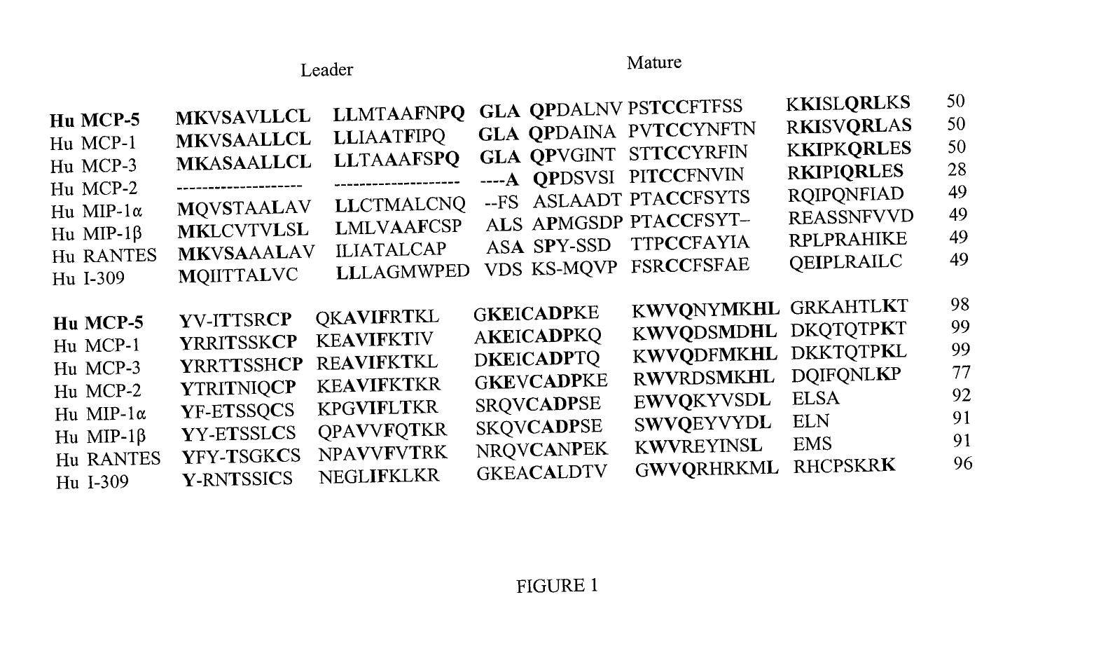

The invention is based upon the isolation of a full length cDNA sequence encoding MCP-5. The deduced amino acid sequence of this cDNA is ninety-eight amino acids in length, of which the first twenty-three N-terminal residues comprise a signal sequence. Manual comparison of the deduced MCP-5 amino acid sequence with sequences of known chemokines in Table 1 and FIG. 1 indicates that it shares 30-64% amino acid identity with other C—C chemokines. The amino acid sequence of MCP-5 appears to be most similar to MCP-1 and MCP-3. The structure of MCP-5 strongly conforms to that of known C—C chemokines in several respects. Similarities include the size of the protein; the position of signal sequence cleavage; the position of the four requisite cysteine residues; and several other amino acids characteristic of C—C chemokines (see FIG. 1). Pairwise comparison of the predicted protein to each of the known C—C chemokines indicates that it is approximately 30% identical to most of these proteins and over 60% identical to MCP-1 and MCP-3 (see Table 1). Dendrogram analysis demonstrates that the MCPs form a sub-family of the C—C chemokines. Despite this clear structural similarity, the functional properties and expression pattern of MCP-5 are distinct from those of MCP-1.

MCP-5 exhibits MCP attractant and activating biological activities in in vitro chemotaxis and calcium flux assays. However, whereas the C—C chemokines have traditionally been characterized by their rapid induction in response to pro-inflammatory stimuli, this does not appear to be the case for MCP-5. The expression of MCP-5 was low in resting PBMC or freshly isolated monocytes and could not be augmented by treatment with LPS or PMA. Nor could MCP-5 expression be induced in endothelial cells or fibroblasts by treatment with TNFa under conditions which would evoke a rapid induction of MCP-1 by these cell types. Furthermore, MCP-5 mRNA is expressed constitutively in a number of normal tissues, in particular small intestine and colon. MCP-5 may play a role in the normal trafficking of leukocytes to these or other tissue sites.

MCP-5 elicits a lower level of chemotaxis by THP-1 cells relative to MCP-1. However, data from calcium flux assays indicates that MCP-1 and MCP-5 interact with a common receptor. Pre-treatment of the THP-1 cells with MCP-1 or MCP-3 blocks the effect of MCP-5 in calcium flux assays, but pre-treatment with MCP-5 does not completely block responsiveness to MCP-1 or MCP-3. Similar calcium flux assay results were obtained when these chemokines were used to treat 293 cells transfected with the human MCP-1 receptor. MCP-5 thus appears to be a weak agonist for the MCP-1 receptor and may interact more strongly with another MCP-5 receptor.

TABLE 1

| | MCP-5 | MCP-1 | MCP-2 | MCP-3 | RANTES | MIP-1α | MIP-1β | I-309 |
|---|---|---|---|---|---|---|---|---|
| MCP-5 | | 64% | 55% | 63% | 32% | 39% | 39% | 30% |
| MCP-1 | 64% | | 61% | 72% | 34% | 38% | 34% | 33% |
| MCP-2 | 55% | 61% | | 59% | 30% | 36% | 33% | 34% |
| MCP-3 | 63% | 72% | 59% | | 34% | 35% | 35% | 37% |
| RANTES | 32% | 34% | 30% | 34% | | 50% | 44% | 22% |
| MIP-1α | 39% | 38% | 36% | 35% | 50% | | 55% | 39% |
| MIP-1β | 39% | 34% | 33% | 35% | 44% | 55% | | 31% |
| I-309 | 30% | 33% | 34% | 37% | 22% | 39% | 31% | |

Amino Acid Identity Among MCP-5 and C-C Chemokines. Percent identity among the chemokines includes the signal sequences, except in the case of MCP-2. Only the sequence of the mature form of the MCP-2 is available for comparison.

Other aspects and advantages of the present invention will be understood upon consideration of the following illustrative examples. Example 1 describes the isolation of a full length MCP-5 cDNA from a human macrophage cDNA library. Example 2 describes experiments examining the pattern of MCP-5 gene expression in various human cell lines and tissues. Example 3 describes the recombinant expression of the MCP-5 gene in mammalian cells and purification of the resulting protein. Example 4 provides a protocol for expression of the MCP-5 gene in prokaryotic cells and purification of the resulting protein. Example 5 provides a protocol for the recombinant production of MCP-5 in yeast. Example 6 describes production of MCP-5 and MCP-5 polypeptide analogs by peptide synthesis or recombinant production methods. Example 7 provides a protocol for generating monoclonal antibodies that are specifically immunoreactive with MCP-5. Examples 8-15 provide protocols for the determination of MCP-5 biological activities. Example 8 compares the effects of MCP-5 and MCP-1 on monocyte chemotaxis in vitro, and Example 9 compares the effects of MCP-5 and MCP-1 on monocyte activation in a calcium flux assay. Examples 10 and 11 provide assays for chemokine effects upon basophils, mast cells, eosinophils, monocytes, macrophages and neutrophils. Examples 12, 13, 14 and 15 provide in vivo assays of tumor growth inhibition, leukocyte activation after intraperitoneal or subcutaneous injection, and myelosuppressive activity. Example 16 describes cloning of another MCP-5 receptor.

### EXAMPLE 1

### Cloning a Full Length cDNA Sequence Encoding MCP-5

A DNA sequence encoding an incomplete fragment of MCP-5 was identified by using the BLAST service of GenBank to compare the coding region of MCP-1 (Matsushima et al., supra) to random sequences in the Expressed Sequence Tags (EST) database of GenBank. The previously uncharacterized EST designated NCBI_ID #118741 was observed to exhibit 60% homology to portions of the genes encoding MCP-1 and MCP-3, but appeared to be truncated. The database description reported that this EST had been cloned from a normal human lung tissue cDNA library using an oligo dT primer. Synthetic oligonucleotides containing sequences complementary to the ends of this EST fragment, e118-F1 (5′-TAT AAG CTT CCT TTC AAC ATG AAA GTC TC, SEQ ID NO: 4) and e118-R2 (5′-TAT TCT AGA TCA TGT CTT TGG TGT GAA CTT TCC GGC CC, SEQ ID NO: 5) were used as primers in a polymerase chain reaction (PCR) to amplify the EST sequence from a different cDNA library, derived from human macrophages [Tjoelker et al., Nature, 374:549-552 (1995)].

Briefly, the cDNA library was prepared as follows. Poly A⁺ RNA was harvested from peripheral blood monocyte-derived macrophages. Double-stranded, blunt-ended cDNA was generated using the Invitrogen Copy Kit (San Diego, Calif.) and BstXI adapters were ligated to the cDNA prior to insertion into the mammalian expression vector, pRc/CMV (Invitrogen). E. coli XL1-Blue bacteria (Stratagene, La Jolla, Calif.) were transformed via electroporation with the plasmid cDNA library and plated onto 986 plates containing 100 μg/ml carbenicillin (approximately 3000 transformants per plate). After overnight growth at 37° C., the bacteria were scraped off of each plate to form 986 bacterial pools. Plasmid DNA was isolated from each of the 986 bacterial pools using the Wizard Miniprep DNA Purification System (Promega, Madison, Wis.).

PCR amplifications were used to screen 60 individual DNA pools to identify those that contained plasmids harboring the MCP-5 cDNA. Each PCR reaction mixture contained 0.2 μg of DNA from a single DNA pool, 1.5 mM MgCl₂, 10 mM Tris pH 8.4, 0.2 mM each dNTP, 10 μg/ml of each primer e118-F1 and e118-R2, and 0.5 μL Taq polymerase (5 U/μl) (Boehringer Mannheim Biochemicals, Indianapolis, Ind.). The reactions were incubated for 4 minutes at 94° C., followed by 35 cycles of denaturation at 94° C. for 15 sec., annealing at 60° C. for 15 sec., and extension at 72° C. for 60 sec. The PCR reaction products were fractionated by electrophoresis through 2% agarose gels (Life Technologies, Inc., Gaithersburg, Md.) in 0.5 X TBE buffer [Sambrook et al., Molecular Cloning: a Laboratory Manual, Cold Spring Harbor, New York: Cold Spring Harbor Laboratory (1987)] and visualized with ethidium bromide. Of the 60 pools screened, 12 produced a brightly staining band at the expected size of 0.3 kilobases, indicating the presence of one or more plasmids containing sequences closely related to the MCP-5 cDNA.

To isolate such related clones, aliquots from six positive plasmid pools were electroporated into E. coli XL1-Blue cells, which were plated and grown overnight on agarose containing 100 μg/ml carbenicillin. Colonies were transferred to nitrocellulose and prepared for hybridization following standard protocols (Sambrook et al., supra).

A radiolabeled MCP-5 probe for screening the filters was prepared from two synthetic oligonucleotides. Oligonucleotide e118-L1 consisted of nucleotides 73 to 135 of SEQ ID NO: 1, and the sequence of oligonucleotide e118-L2 was complementary to nucleotides 184 to 121 of SEQ ID NO: 1. The 15 nucleotides at the 3′ termini of these oligonucleotides were annealed to each other in a reaction containing 0.5 μg of each oligonucleotide in addition to DATP, dGTP, and Klenow buffer from the Random Prime DNA Labeling Kit (Boehringer Mannheim). The reaction mixture was momentarily heated to 65° C. and allowed to cool to 37° C. Radiolabeled dCTP and dTTP (DuPont/New England Nuclear, Boston, Mass.) were added to the reaction and incorporated into the paired oligonucleotides by Klenow polymerase (BMB). Unincorporated nucleotides were removed by passing the reaction product through a Sephadex G-25 Quick Spin Column (BMB).

The labeled probe was hybridized to the filters and washed according to standard protocols (Sambrook et al., supra). Hybridization was detected by exposing Kodak XAR-5 film to the filters for 3 hours at −80° C. with an intensifying screen (Lightening Plus, DuPont, Del.). Cultures were grown from the hybridizing colonies, and plasmid DNA was isolated using the Wizard DNA Purification Kit (Promega). The plasmid inserts were sequenced using an Applied Biosystems Automated DNA Sequencer (Model 373, Foster City, Calif.).

An 860-bp clone containing a complete coding sequence (SEQ ID NO: 3) was recovered and the protein thereby encoded was designated MCP-5. Nucleotides 58 to 358 of SEQ ID NO: 3 correspond to the EST sequence NCBI_ID #118741, with the following exceptions: NCBI_ID#118741 contained an additional ‘T’ inserted after nucleotide 70 of SEQ ID NO: 3; NCBI_ID#118741 contained an additional ‘G’ after nucleotide 282 of SEQ ID NO: 3; NCBI_ID#118741 contained two additional ‘G's after nucleotide 344 of SEQ ID NO: 3; and NCBI_ID#118741 contained a ‘T’ in place of the ‘C’ present at nucleotide 353 in SEQ ID NO: 3. These discrepancies between the EST sequence and nucleotides 58 to 358 of SEQ ID NO: 3 result in two frame shifts, which would produce differing predicted protein sequences.

### EXAMPLE 2

### MCP-5 Gene Expression Pattern in Cell Lines and Tissues

The pattern of MCP-5 mRNA expression was examined through Northern blotting of mRNA extracted from various human tissues and cell lines.

A. MCP-5 Gene Expression in Human Tissues

The expression of MCP-5 RNA in various human tissues was examined by probing a Northern blot with a fragment of the MCP-5 cDNA. The fragment was generated in a PCR reaction contained 0.2 μg of the full-length MCP-5 clone (described in Example 1) and the following primers e118-5B (SEQ ID NO: 6) and e118-4R (SEQ ID NO: 7):

| | |
|---|---|
| e118-5B: | 5′-AAT CGG ATC CGG CGG AAC AGC GAG AGG |
| | AG-3′ |
| e118-4R: | 5′-CAG CAA CCT ACT TGC TCA AG-3′ |

Primer e118-5B includes nucleotides 9 to 27 of SEQ ID NO: 3 adjacent to a BamHI restriction endonuclease site, and primer e118-4R consists of the nucleotides complementary to nucleotides 600 to 619 of SEQ ID NO: 3. The reaction conditions were as described in Example 1.

The PCR product was run on a 2% agarose gel in TAE buffer (Sambrook et al., supra) and visualized with ethidium bromide. A single intense band at the expected size of 0.6 kb was excised, electroeluted in TAE buffer, and precipitated in ethanol according to standard protocols (Sambrook et al., supra). The fragment was labeled with the Random Primed DNA Labeling Kit (BMB) and hybridized to a Multiple Tissue Northern Blot (Clontech, Palo Alto, Calif.) according to the manufacturer's recommendations.

The highest MCP-5 expression was seen in small intestine and colon, with lower expression levels observed in the lung, heart, placenta, and thymus. Upon overexposure of the film, very low expression was detectable in liver, skeletal muscle, kidney, pancreas, prostate, testis, uterus, and peripheral blood leukocytes, but could not be detected in brain and spleen.

B. MCP-5 Gene Expression During Macrophage Maturation

MCP-5 expression by human monocytes and differentiated macrophages was examined. Human monocytes from a single donor were isolated by adherence to plastic and cultured for 8 hours in the presence or absence of 100 ng/ml LPS, or cultured in the absence of stimulation for 6 days (under these conditions the monocytes differentiate into macrophages, Tjoelker, supra).

A Northern blot of RNA (20 μg per lane) isolated from these cells was prepared and probed using the probe for MCP-5 described in section A of this example. MCP-5 was expressed at low levels in unstimulated monocytes and in differentiated macrophages. Its expression was not augmented by treating the monocytes with LPS (a potent inducer of MCP-1).

The expression of MCP-5 was low in resting PBMC or freshly isolated monocytes and could not be augmented by treatment with LPS. Nor could MCP-5 expression be induced in endothelial cells, epithelial cells (A549), or fibroblasts (IMR 90) by treatment with TNFα. Under the same conditions, a rapid and sustained increase in MCP-1 expression by these cell types was observed.

### EXAMPLE 3

### Production of Recombinant MCP-5 in Mammalian Cells

Recombinant MCP-5 was produced by stably transfecting the MCP-5 cDNA into CHO cells. PCR was used to amplify nucleotides 9 to 383 of SEQ ID NO: 3, which includes 67 bp of 5′ non-coding and 11 bp of 3′ non-coding sequence. The template used for the reaction was the full-length MCP-5 cDNA clone, and the primers were e118-5B (described above in Example 2A) and e118-term (5′-CCA TGA ATT CGG TAG CAG AGT TCA AGT C-3′, SEQ ID NO: 8) which includes the sequence complementary to nucleotides 366 to 383 of SEQ ID NO: 3 adjacent to an EcoRI restriction endonuclease site. The reaction conditions were similar to those described in Example 1, except that the extension portion of the cycle was reduced to 30 sec.

The PCR product was purified by electrophoresis and precipitation, as described above in Example 2B. The resulting fragment was digested with BamHI and EcoRI and inserted into the vector pDC1, which had been digested with BglII and EcoRI. This vector contains the CMV immediate early promoter adjacent to the cloning site to facilitate expression of the insert. It also contains the bacterial beta-lactamase gene and the murine dihydrofolate reductase (DHFR) gene to allow selection of the plasmid in bacterial and mammalian cells, respectively (Sambrook et al., supra).

This fragment was cloned into the vector pDC1. For electroporation, 10⁷ CHO cells were washed, resuspended in 1 ml PBS, mixed with 30 μg of linearized plasmid, and transferred to a 0.4 cm cuvette. The suspension was electroporated with a Biorad Gene Pulser (Richmond, Calif.) at 290 volts, 960 μF. Transformants were selected by growth in α⁻ medium lacking hypoxanthine and thymidine (Gibco Alpha Cat. No. 12000 plus 10% dialyzed fetal bovine serum, 2 mM L-glutamine, 1 mM sodium pyruvate, 100 units/ml penicillin, and 100 μg/ml streptomycin). Cells from several hundred transformed colonies were pooled and replated in α⁻ medium containing 20 nM methotrexate. Colonies surviving this round of selection were isolated and expanded.

Clones were grown to approximately 90% confluence in α⁻ medium, then grown 3-4 days longer in α⁻ medium containing 0.5% serum. The supernatants were brought to pH 6.8 and loaded onto Heparin-Sepharose columns (Pharmacia, Piscataway, N.J.). Columns were washed with 0.2 M NaCl, and the chemokine was eluted with 0.6 M NaCl. The eluted material was fractionated by SDS-PAGE (18% acrylamide, Tris-glycine gel, NOVEX, San Diego, Calif.) and transferred to a PVDF membrane. A 6.4 kD band unique to the transfectants and absent from the untransfected controls corresponded to the expected size of MCP-5. This band was excised and the N-terminus sequenced on an Applied Biosystems Model 473A, Foster City, Calif. automated sequencer. The results of sequencing the first nine N-terminal amino acids indicated that the purified protein was the mature form of MCP-5, beginning at Gln24. This cleavage site is consistent with the processing sites of MCP-1 and MCP-3.

### EXAMPLE 4

### Production of Recombinant MCP-5 in Bacteria

The DNA sequence encoding part of the leader sequence and the mature form of the protein was amplified by PCR and cloned into the vector pGEX-3X (Pharmacia, Piscataway, N.J.). The pGEX vector is designed to produce a fusion protein comprising glutathione-S-transferase (GST), encoded by the vector, and a protein encoded by a DNA fragment inserted into the vector's cloning site. The primers for the PCR were e118-term (described above in Example 3) and 118-TF2 (5′-TAT CGG ATC CTG GTT CCG CGT CAG GGA CTT GCT CAG CCA G-3′, SEQ ID NO: 9), which includes a BamHI restriction site, a thrombin cleavage site [Chang, Eur J. Biochem., 151:217 (1985)], and nucleotides 58 to 76 of SEQ ID NO: 1. The resultant PCR product is digested with BamHI and EcoRI and inserted into a pGEX-3X plasmid digested with BglII and EcoRI. Treatment of the recombinant fusion protein with thrombin or factor Xa (Pharmacia, Piscataway, N.J.) is expected to cleave the fusion protein, releasing the chemokine from the GST portion.

The pGEX-3X/MCP-5 construct was transformed into E. coli XL-1 Blue cells (Stratagene, La Jolla Calif.), and individual transformants were isolated and grown. Plasmid DNA from individual transformants was purified and partially sequenced using an automated sequencer to confirm the presence of the desired MCP-5 gene insert in the proper orientation.

Induction of the GST/MCP-5 fusion protein was achieved by growing the transformed XL-1 Blue culture at 37° C. in LB medium (supplemented with carbenicillin) to an optical density at wavelength 600 nm of 0.4, followed by further incubation for 4 hours in the presence of 0.5 mM Isopropyl β-D-Thiogalactopyranoside (Sigma Chemical Co., St. Louis Mo.).

The fusion protein, produced as an insoluble inclusion body in the bacteria, was purified as follows. Cells were harvested by centrifugation; washed in 0.15 M NaCl, 10 mM Tris, pH 8, 1 mM EDTA; and treated with 0.1 mg/ml lysozyme (Sigma Chemical Co.) for 15 minutes at room temperature. The lysate was cleared by sonication, and cell debris was pelleted by centrifugation for 10 minutes at 12,000 X g. The fusion protein-containing pellet was resuspended in 50 mM Tris, pH 8, and 10 mM EDTA, layered over 50% glycerol, and centrifuged for 30 min. at 6000 X g. The pellet was resuspended in standard phosphate buffered saline solution (PBS) free of Mg++and Ca++.

The fusion protein was further purified by fractionating the resuspended pellet in a denaturing SDS polyacrylamide gel (Sambrook et al., supra). The gel was soaked in 0.4 M KCl to visualize the protein, which was excised and electroeluted in gel-running buffer lacking SDS. The resultant protein was injected into rabbits to raise anti-MCP-5 antibodies, following standard protocols (Sambrook et al., supra, Chapter 18). The protein may also be used to generate monoclonal antibodies as described in Example 7.

Mature MCP-5 protein may be produced in a similar fashion. PCR amplification is performed using primers e118-term and e118-TF3 (5′-TAT CGG ATC CTG GTT CCG CGT CAG CCA GAT GCA CTC AAC GTC-3′, SEQ ID NO: 10), which includes a BamHI restriction site, a thrombin cleavage site, and nucleotides 70 to 87 of SEQ ID NO: 1. The resultant PCR product is cleaved with BamHI and EcoRI and inserted into a pGEX-3X plasmid digested with BglII and EcoRI, which is then transformed into bacteria and grown as described above. The fusion protein is subjected to thrombin digestion to cleave the GST from the mature MCP-5 protein. The digestion reaction (20-40 ug fusion protein, 20-30 units human thrombin (4000 U/ mg (Sigma) in 0.5 ml PBS) is incubated 16-48 hrs. at room temperature and loaded on a denaturing SDS-PAGE gel to fractionate the reaction products. The gel is soaked in 0.4 M KCl to visualize the protein bands. The identity of the protein band corresponding to the expected molecular weight of MCP-5 may be confirmed by partial amino acid sequence analysis using an automated sequencer (Applied Biosystems Model 473A, Foster City, Calif.).

Alternatively, the DNA sequence encoding the predicted mature MCP-5 protein may be cloned into a plasmid containing a desired promoter and, optionally, a leader sequence [see, e.g., Better et al., Science, 240:1041-43 (1988)]. The sequence of this construct may be confirmed by automated sequencing. The plasmid is then transformed into E. coli strain MC1061 using standard procedures employing CaCl₂ incubation and heat shock treatment of the bacteria (Sambrook et al., supra). The transformed bacteria are grown in LB medium supplemented with carbenicillin, and production of the expressed protein is induced by growth in a suitable medium. If present, the leader sequence will effect secretion of the mature MCP-5 protein and be cleaved during secretion.

The secreted recombinant protein is purified from the bacterial culture media by the method described above in Example 3 or, e.g., by adapting methods previously described for the purification of recombinantly produced RANTES chemokine [Kuna et al., J. Immunol., 149:636-642 (1992)], MGSA chemokine [Horuk et al., J. Biol. Chem. 268:541-46 (1993)], and IP-10 chemokine (expressed in insect cells) [Sarris et al., J. Exp. Med., 178:1127-1132 (1993)].

### EXAMPLE 5

### Recombinant Production of MCP-5 in Yeast

Exemplary protocols for the recombinant expression of MCP-5 in yeast and for the purification of the resulting recombinant protein follow.

The coding region of the MCP-5 cDNA is amplified by PCR. A DNA encoding the yeast pre-pro-alpha leader sequence is amplified from yeast genomic DNA in a PCR reaction using one primer containing nucleotides 1-20 of the alpha mating factor gene and another primer complementary to nucleotides 255-235 of this gene [Kujan and Herskowitz, Cell, 30:933-943 (1982)]. The pre-pro-alpha leader coding sequence and MCP-5 coding sequence fragments are ligated into a plasmid containing the yeast alcohol dehydrogenase (ADH2) promoter, such that the promoter directs expression of a fusion protein consisting of the pre-pro-alpha factor fused to the mature MCP-5 polypeptide. As taught by Rose and Broach, Meth. Enz. 185:234-279, D. Goeddel, ed., Academic Press, Inc., San Diego, Calif. (1990), the vector further includes an ADH2 transcription terminator downstream of the cloning site, the yeast “2-micron” replication origin, the yeast leu-2d gene, the yeast REP1 and REP2 genes, the E. coli beta-lactamase gene, and an E. coli origin of replication. The beta-lactamase and leu-2d genes provide for selection in bacteria and yeast, respectively. The leu-2d gene also facilitates increased copy number of the plasmid in yeast to induce higher levels of expression. The REP1 and REP2 genes encode proteins involved in regulation of the plasmid copy number.

The DNA construct described in the preceding paragraph is transformed into yeast cells using a known method, e.g., lithium acetate treatment [Stearns et al., Meth. Enz., supra, pp. 280-297]. The ADH2 promoter is induced upon exhaustion of glucose in the growth media [Price et al., Gene, 55:287 (1987)]. The pre-pro-alpha sequence effects secretion of the fusion protein from the cells. Concomitantly, the yeast KEX2 protein cleaves the pre-pro sequence from the mature MCP-5 chemokine [Bitter et. al., Proc. Natl. Acad. Sci. USA, 81:5330-5334 (1984)].

Alternatively, MCP-5 is recombinantly expressed in yeast using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol.

The secreted recombinant MCP-5 is purified from the yeast growth medium by, e.g., the methods used to purify MCP-5 from bacterial and mammalian cell supernatants (see Examples 3 and 4 above).

### EXAMPLE 6

### Production of MCP-5 Analogs

Recombinant techniques such as those described in the preceding examples may be used to prepare MCP-5 polypeptide analogs. More particularly, polynucleotides encoding MCP-5 are modified to encode polypeptide analogs of interest using well-known techniques, e.g., site-directed mutagenesis and polymerase chain reaction. See generally Sambrook et al., supra, Chapter 15. The modified polynucleotides are expressed recombinantly, and the recombinant polypeptide analogs are purified as described in the preceding examples.

Residues critical for MCP-5 activity are identified, e.g., by homology to other C—C chemokines and by substituting alanines for the native MCP-5 amino acid residues. Cysteines are often critical for the functional integrity of proteins because of their capacity to form disulfide bonds. To determine whether any of the four cysteines in MCP-5 is critical for enzyme activity, each cysteine is mutated individually to a serine.

C-terminal deletions are prepared, e.g., by digesting the 3′ end of the MCP-5 coding sequence with exonuclease III for various amounts of time and then ligating the shortened coding sequence to plasmid DNA encoding stop codons in all three reading frames. N-terminal deletions are prepared in a similar manner by digesting the 5′ end of the coding sequence and then ligating the digested fragments into a plasmid containing a promoter sequence and an initiating methionine immediately upstream of the promoter site. These N-terminal deletion analogs may also be expressed as fusion proteins.

Alternatively, MCP-5 polypeptide analogs may also be prepared by chemical peptide synthesis using techniques that have been used successfully for the production of other chemokines such as IL-8 [Clark-Lewis et al., J. Biol Chem., 266:23128-34 (1991)] and MCP-1. Such methods are advantageous because they are rapid, reliable for short sequences such as chemokines, and allow the selective introduction of novel, unnatural amino acids and other chemical modifications.

The chemoattractant and/or cell-activation properties of MCP-5 polypeptide analogs on one or more types of cells involved in the inflammatory process, (e.g., T lymphocytes, monocytes, macrophages, basophils, eosinophils, neutrophils, mast cells, endothelial cells, epithelial cells or others) are assayed by art-recognized techniques that have been used for assaying such properties of numerous other chemokines, such as those described in Examples 8-15 below.

### EXAMPLE 7

### Preparation of Monoclonal Antibodies to MCP-5

A protocol is described for generating monoclonal antibodies to MCP-5. A mouse is injected periodically with recombinant MCP-5 (e.g., 10-20 μg emulsified in Freund's Complete Adjuvant) obtained as described in any of Examples 3 through 6. The mouse is given a final pre-fusion boost of MCP-5 in PBS, and four days later the mouse is sacrificed and its spleen removed. The spleen is placed in 10 ml serum-free RPMI 1640, and a single cell suspension is formed by grinding the spleen between the frosted ends of two glass microscope slides submerged in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspension is filtered through sterile 70-mesh Nitex cell strainer (Becton Dickinson, Parsippany, N.J.), and is washed twice by centrifuging at 200 g for 5 minutes and resuspending the pellet in 20 ml serum-free RPMI. Splenocytes taken from three naive Balb/c mice are prepared in a similar manner and used as a control. NS-1 myeloma cells, kept in log phase in RPMI with 11% fetal bovine serum (FBS) (Hyclone Laboratories, Inc., Logan, Utah) for three days prior to fusion, are centrifuged at 200 g for 5 minutes, and the pellet is washed twice as described in the foregoing paragraph.

One ×10⁸ spleen cells are combined with 2.0×10⁷ NS-1 cells and centrifuged, and the supernatant is aspirated. The cell pellet is dislodged by tapping the tube, and 1 ml of 37° C. PEG 1500 (50% in 75 mM Hepes, pH 8.0) (Boehringer Mannheim) is added with stirring over the course of 1 minute, followed by the addition of 7 ml of serum-free RPMI over 7 minutes. An additional 8 ml RPMI is added and the cells are centrifuged at 200 g for 10 minutes. After discarding the supernatant, the pellet is resuspended in 200 ml RPMI containing 15% FBS, 100 μM sodium hypoxanthine, 0.4 μM aminopterin, 16 μM thymidine (HAT) (Gibco), 25 units/ml IL-6 (Boehringer Mannheim) and 1.5×10⁶ splenocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusion is screened by ELISA, testing for the presence of mouse IgG binding to MCP-5 as follows. Immulon 4 plates (Dynatech, Cambridge, Mass.) are coated for 2 hours at 37° C. with 100 ng/well of MCP-5 diluted in 25mM Tris, pH 7.5. The coating solution is aspirated and 200 ul/well of blocking solution [0.5% fish skin gelatin (Sigma) diluted in CMF-PBS] is added and incubated for 30 min. at 37° C. Plates are washed three times with PBS with 0.05% Tween 20 (PBST) and 50 μl culture supernatant is added. After incubation at 37° C. for 30 minutes, and washing as above, 50 μl of horseradish peroxidase conjugated goat anti-mouse IgG(fc) (Jackson ImmunoResearch, West Grove, Pa.) diluted 1:3500 in PBST is added. Plates are incubated as above, washed four times with PBST, and 100 μL substrate, consisting of 1 mg/ml o-phenylene diamine (Sigma) and 0.1 μl/ml 30% H₂O₂ in 100 mM Citrate, pH 4.5, are added. The color reaction is stopped after 5 minutes with the addition of 50 μl of 15% H₂SO₄. A₄₉₀ is read on a plate reader (Dynatech).

Selected fusion wells are cloned twice by dilution into 96-well plates and visual scoring of the number of colonies/well after 5 days. The monoclonal antibodies produced by hybridomas are isotyped using the Isostrip system (Boehringer Mannheim, Indianapolis, Ind.).

### EXAMPLE 8

### Effects of MCP-5 on Transmigration

An in vitro chemotaxis assay was used to evaluate the effects of recombinant MCP-5 on transmigration, and to compare its activity to that of recombinant MCP-1 (Matsushima et al., supra) similarly produced and purified in CHO cells. The chemotactic response of the human monocyte-derived cell line THP-1 (ATCC Accession No. TIB202) was measured in a transwell assay, as described by Casale et al., Am. J. Resp. Cell Mol. Biol., 7:112-117 (1992). Transmigration chambers (polycarbonate membrane, 8 um pore) were purchased from Costar (Cambridge, Mass.). Briefly, 10⁶ cells labelled with ⁵¹Cr were resuspended in RPMI medium and added to the upper chamber, 0.5 ml of RPMI plus the chemokine to be tested was added to the lower chamber. After incubation of 60-90 minutes at 37° C., cells that had migrated through the filter and adhered to the lower side were washed off with 5 mM EDTA in PBS and added to those cells that had fallen into the lower chamber.

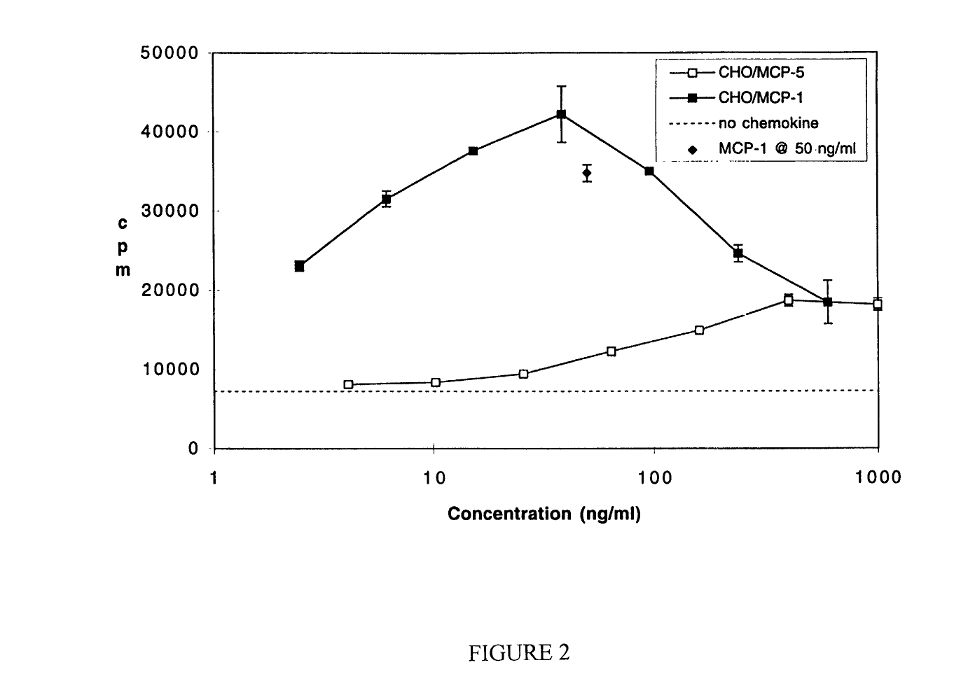

The resulting data are shown in FIG. 2. The open squares show chemotactic response to MCP-5, while the filled squares show the response to MCP-1. The filled diamond indicates the response to commercially obtained MCP-1 (Peprotech, Rocky Hill, N.J.) at a concentration of 50 ng/ml. The dotted line indicates the baseline cpm value with no added chemokine.

The THP-1 cells exhibited a distinct response to MCP-5, bet the response failed to peak at concentrations up to 1 μg/ml, suggesting that MCP-5 interacts weakly with a receptor on this cell line. In contrast, MCP-1 induced a dose response curve typical of many chemokines, with a strong maximal response at 40-80 ng/ml.

### EXAMPLE 9

### Effect of MCP-5 on Activation of Monocytic Cells

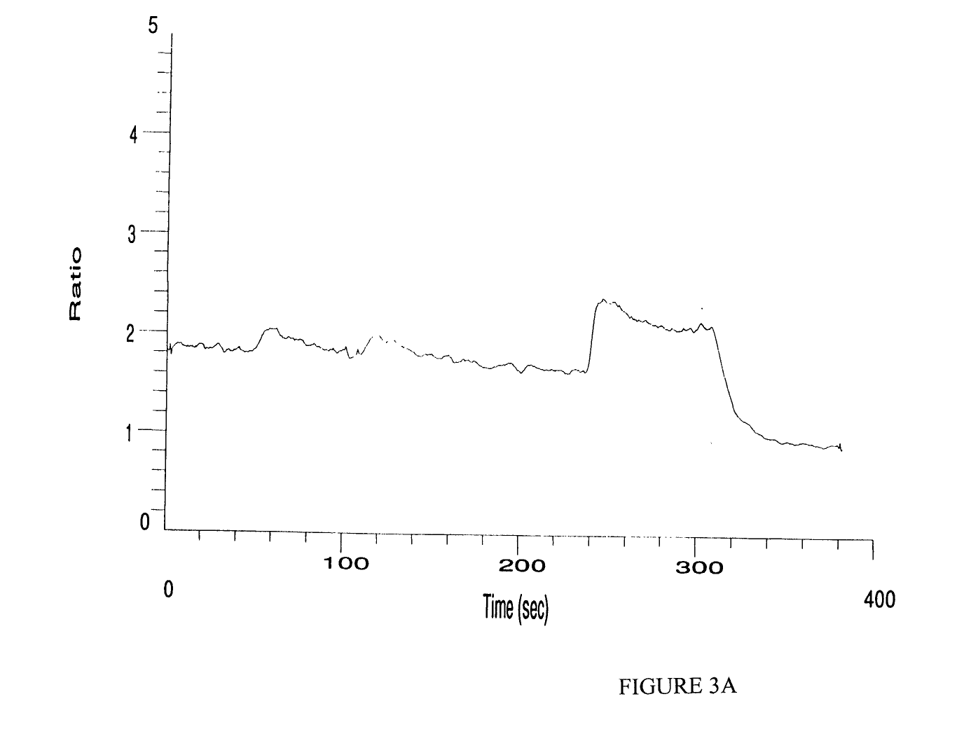

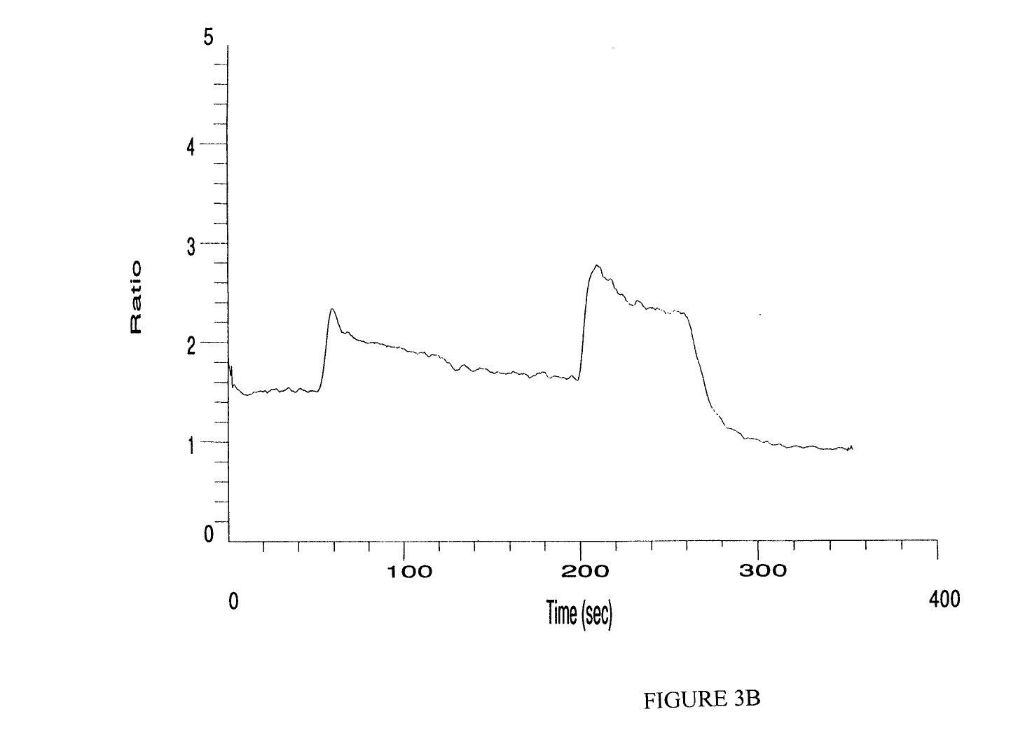

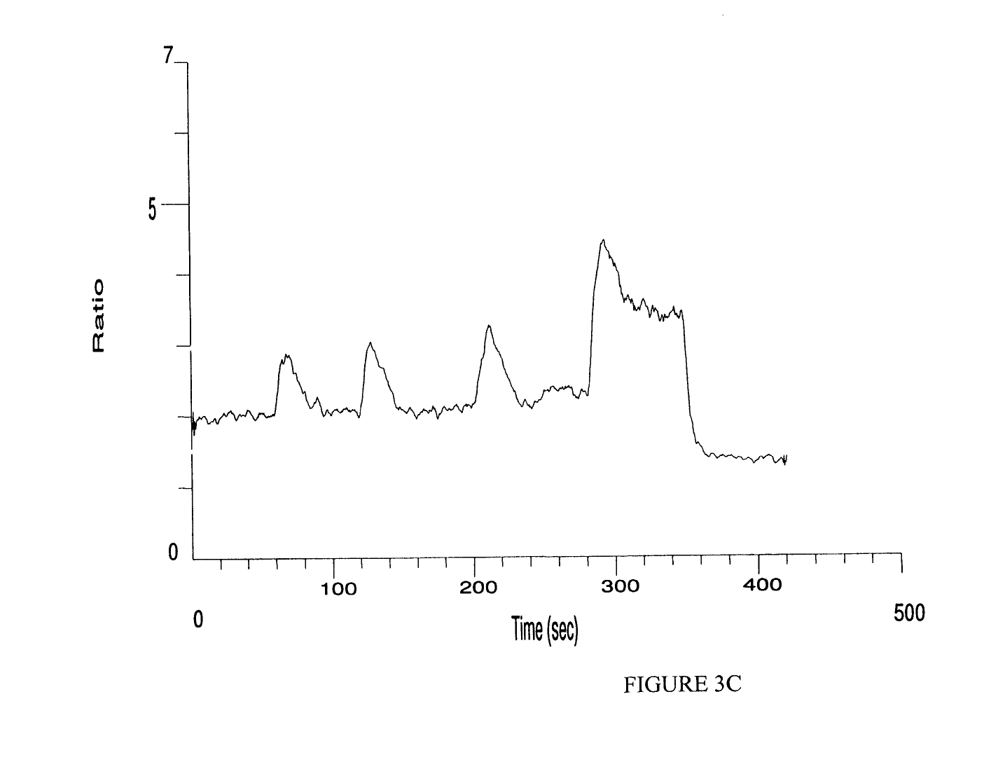

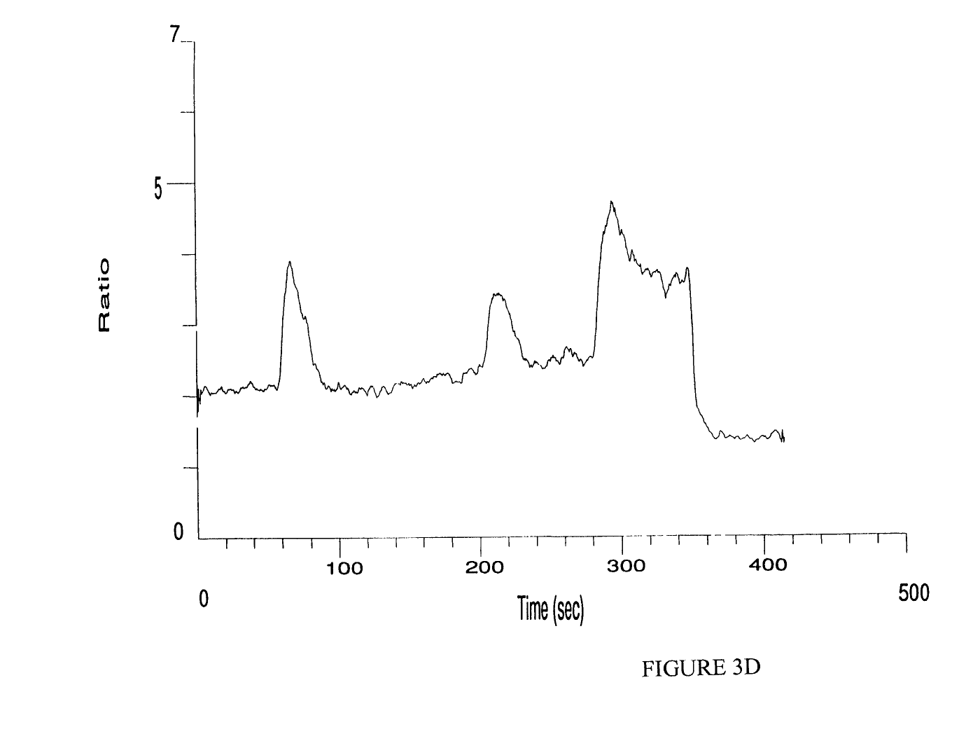

The interaction of MCP-1 and MCP-5 with monocytic cells and with the MCP-1 receptor was further studied through a calcium flux assay. Intracellular calcium fluxes were monitored by incubating cells in 1 ml complete media containing 1 μM Fura-1/AM (Molecular Probes, Eugene, Oreg.) for 30 min. at room temperature. Cells were washed once with PBS and resuspended at a density of ˜10⁶ cells/ml. Two ml of suspended THP-1 cells were placed in a continuously stirred cuvette at 37 C in a fluorimeter (AMINCO-Bowman Series 2, Rochester, N.Y.). The chemokines were sequentially added to the cells. To provide controls, the cells were then treated with ionomycin (1 μg/ml, Sigma Chemical Co., St. Louis, Mo.) to induce the maximum possible calcium increase, and with 1 mM ethylene glycol-bis(β-aminoethyl ether)N,N,N′,N′-tetraacetic acid (EGTA) to chelate the available calcium. The change in intracellular calcium concentration in response to added chemokines is reflected by a change in fluorescence of the treated cells. Fluorescence was monitored at 510 nm emission wavelength while switching between excitation wavelengths of 340 nm and 380 nm every 0.5 sec. The data, expressed as the ratio of the 340 nm to 380 nm excitation spectra, are shown in FIG. 3A (in which MCP-5 was added at 50 sec., followed by MCP-1 at 110 sec., ionomycin at 240 sec., and EGTA at 310 sec.) and FIG. 3B (in which MCP-1 was added at 50 sec., followed by MCP-5 at 120 sec., ionomycin at 200 sec., and EGTA at 260 sec.).

THP-1 cells underwent a significant calcium flux in response to MCP-5 (see FIG. 3A), but this effect was blocked in cells that had been previously activated with MCP-1 (see FIG. 3B). In contrast, the response to MCP-1 was diminished, but not blocked, by pre-treatment of the cells with MCP-5. Similar results were obtained with freshly isolated peripheral blood mononuclear cells and monocytes. These results implied that MCP-5 either interacted with a subset of receptors recognized by MCP-1 or transduced a sub-optimal signal through the MCP-1 receptor, allowing a further response upon binding of MCP-1.

To distinguish between these two possibilities, MCP-1 and MCP-5 were tested on a human embryonic kidney cell line 293 that had been transfected with the MCP-1 receptor CCR2-B (Charo et al., supra). In addition to the ionomycin and EGTA controls, the cells were also treated with thrombin (Sigma, St. Louis, Mo.) to indicate the response due to activation of the native thrombin receptors on these cells. Results are shown in FIG. 3C (in which MCP-5 was added at 60 sec., MCP-1 at 120 sec., thrombin at 200 sec., ionomycin at 280 sec., and EGTA at 350 sec.) and FIG. 3D (in which MCP-1 was added at 60 sec., MCP-5 at 120 sec., thrombin at 200 sec., ionomycin at 280 sec., and EGTA at 350 sec.). The response of 293 cells transfected with the MCP-1 receptor to MCP-1 and MCP-5 was similar but more pronounced than the response of THP-1 cells, suggesting that MCP-1 and MCP-5 interact with different efficacy through a single receptor. Untransfected 293 cells gave no response to either of these chemokines. Thus, MCP-5 appears to be a weak agonist for the MCP-1 receptor.

### EXAMPLE 10

### Assay of MCP-5 Effects upon Basophils, Mast Cells, and Eosinophils

The effect of MCP-5 upon basophils, mast cells, and eosinophils is assayed, e.g., by methods described by Weber et al., J. Immunol., 154:4166-4172 (1995) for the assay of MCP-1/2/3 activities. In these methods, changes in free cytosolic calcium and release of proinflammatory mediators (such as histamine and leukotriene) are measured. Blocking chemokine-mediated activation of these cell types has implications in the treatment of late-phase allergic reactions, in which secretion of proinflammatory mediators plays a significant role [Weber et al., supra].

### EXAMPLE 11

### Assay of Chemoattractant and Cell-Activation Properties of MCP-5 on Human Monocytes/Macrophages and Human Neutrophils

The effects of MCP-5 upon human monocytes/macrophages or human neutrophils is evaluated, e.g., by methods described by Devi et al., J. Immunol., 153:5376-5383 (1995) for evaluating murine TCA3-induced activation of neutrophils and macrophages. Indices of activation measured in such studies include increased adhesion to fibrinogen due to integrin activation, chemotaxis, induction of reactive nitrogen intermediates, respiratory burst (superoxide and hydrogen peroxide production), and exocytosis of lysozyme and elastase in the presence of cytochalasin B. As discussed by Devi et al., these activities correlate to several stages of the leukocyte response to inflammation. This leukocyte response, reviewed by Springer, Cell, 76:301-314 (1994), involves adherence of leukocytes to endothelial cells of blood vessels, migration through the endothelial layer, chemotaxis toward a source of chemokines, and site-specific release of inflammatory mediators. The involvement of MCP-5 at any one of these stages provides an important target for clinical intervention by modulating the inflammatory response.

### EXAMPLE 12

### MCP-5 In Vivo Tumor Growth Inhibition Assay

Tumor growth-inhibition properties of MCP-5 are assayed, e.g., by modifying the protocol described by Laning et al., J. Immunol., 153:4625-4635 (1994) for assaying the tumor growth-inhibitory properties of murine TCA3. An MCP-5-encoding cDNA is transfected by electroporation into the myeloma-derived cell line J558 (American Type Culture Collection, Rockville, Md.). Transfectants are screened for MCP-5 production by standard techniques such as ELISA (enzyme-linked immunoadsorbant assay) using a monoclonal antibody generated against MCP-5 as detailed in Example 7. A bolus of 10 million cells from an MCP-5-producing clone is injected subcutaneously into the lower right quadrant of BALB/c mice. For comparison, 10 million non-transfected cells are injected into control mice. The rate and frequency of tumor formation in the two groups is compared to determine efficacy of MCP-5 in inhibiting tumor growth. The nature of the cellular infiltrate subsequently associated with the tumor cells is identified by histologic means. In addition, recombinant MCP-5 (20 ng) is mixed with non-transfected J558 cells and injected (20 ng/day) into tumors derived from such cells, to assay the effect of MCP-5 administered exogenously to tumor cells.

### EXAMPLE 13

### Intraperitoneal Injection Assay

The cells which respond to MCP-5 in vivo are determined through injection of 1-100 ng of purified MCP-5 into the intraperitoneal cavity of mice, as described by Luo et al., J. Immunol., 153:4616-4624 (1994). Following injection, leukocytes are isolated from peripheral blood and from the peritoneal cavity and identified by staining with the Diff Quick kit (Baxter, McGraw, Ill.). The profile of leukocytes is measured at various times to assess the kinetics of appearance of different cell types. In separate experiments, neutralizing antibodies directed against MCP-5 (Example 7) are injected along with MCP-5 to confirm that the infiltration of leukocytes is due to the activity of MCP-5.

### EXAMPLE 14

### In vivo Activity Assay—Subcutaneous Injection

The chemoattractant properties of MCP-5 are assayed in vivo by adapting the protocol described by Meurer et al., J. Exp. Med., 178:1913-1921 (1993). Recombinant MCP-5 (10-500 pmol/site) is injected intradermally into a suitable mammal, e.g., dogs or rabbits. At times of 4 to 24 hours, cell infiltration at the site of injection is assessed by histologic methods. The presence of MCP-5 is confirmed by immunocytochemistry using antibodies directed against MCP-5. The nature of the cellular infiltrate is identified by staining with Baxter's Diff Quick kit.

### EXAMPLE 15

### In Vivo Myelosuppression Activity Assay

The myelosuppressive activity of MCP-5 is assayed by injection of MCP-5 into mice, e.g., as described by Maze et al., J. Immunol., 149:1004-1009 (1992) for the measurement of the myelosuppressive action of MIP-1α. A single dose of 0.2 to 10 ug of recombinant MCP-5 is intravenously injected into C3H/HeJ mice (Jackson Laboratories, Bar Harbor Me.). The myelosuppressive effect of the chemokine is determined by measuring the cycling rates of myeloid progenitor cells in the femoral bone marrow and spleen. The suppression of growth and division of progenitor cells has clinical implications in the treatment of patients receiving chemotherapy or radiation therapy. The myeloprotective effect of such chemokine treatment has been demonstrated in pre-clinical models by Dunlop et al., Blood, 79:2221 (1992).

### EXAMPLE 16

### Cloning of an Additional MCP-5 Receptor

DNA encoding an additional MCP-5 receptor is cloned by adapting procedures previously described for isolation of the IL-8 receptor gene in Holmes et al., supra, and isolation of the MCP-1 receptor gene in Charo et al., supra.

A cDNA library is prepared, preferably from cells that respond to MCP-5 by chemotaxis and activation. Radiolabelled MCP-5 can also be used to identify cell types which express high levels of receptor for MCP-5. Cells which do not respond to MCP-1 or MCP-3, or cells which show a different pattern of receptor desensitization in response to these ligands (compared to that seen for the cloned MCP-1 receptor) are of particular interest. Pools of transfected clones in the cDNA library are screened for binding of radiolabelled MCP-5 by autoradiography. Positive pools are successively subfractionated and rescreened until individual positive clones are obtained.

Alternatively, a degenerate PCR strategy may be used in which the sequences of the PCR primers are based on conserved regions of the sequences of known chemokine receptors. The primers may or may not be biased towards the sequence encoding the MCP-1 receptor with which MCP-5 interacts. To increase the chance of isolating an MCP-5 receptor, the template DNA used in the reaction may be cDNA derived from a cell type responsive to MCP-5.

While the present invention has been described in terms of specific embodiments, it is understood that variations and modifications will occur to those skilled in the art. Accordingly, only such limitations as appear in the appended claims should be placed on the invention.

What is claimed is:

1. A purified polynucleotide encoding the monocyte chemotactic protein-5 (MCP-5) amino acid sequence of SEQ ID NO: 2.

2. The polynucleotide of claim 1 which is a DNA.

3. The DNA of claim 2 comprising a nucleotide sequence consisting of the nucleotide sequence set forth in SEQ ID NO: 1.

4. A purified polynucleotide encoding amino acids 1 to 75 of SEQ ID NO: 2.

5. The polynucleotide of claim 4 which is a DNA.

6. The DNA of claim 5 comprising a nucleotide sequence consisting of nucleotides 70 to 297 of SEQ ID NO: 1.

7. A purified polynucleotide encoding a full-length MCP-5 which hybridizes under stringent conditions to the complementary strand of the DNA of SEQ ID NO: 1.

8. The polynucleotide of claim 7 which is a DNA.

9. A vector comprising the DNA of claim 2, 3, 5, 6 or 8.

10. The vector of claim 9 that is an expression vector, wherein the DNA is operatively linked to an expression control DNA sequence.

11. A host cell stably transformed or transfected with the DNA of claim 2, 3, 5, 6 or 8 in a manner allowing the expression in said host cell of MCP-5.

12. A method for producing MCP-5 comprising culturing the host cell of claim 11 in a nutrient medium and isolating MCP-5 from said host cell or said nutrient medium.

13. A purified polypeptide produced by the method of claim 12.

14. A purified polypeptide comprising the MCP-5 amino acid sequence of SEQ ID NO: 2.

15. A purified polypeptide comprising MCP-5 amino acids 1 to 75 of SEQ ID NO: 2.

16. A hybridoma cell line producing a monoclonal antibody that is specifically reactive with the polypeptide of claim 15.

17. The monoclonal antibody produced by the hybridoma of claim 16.
